(19) United States
(12) Patent Application Publication
(10) Pub. No.: US 2010/0316743 A1
(43) Pub. Date: Dec. 16, 2010

(54) INHIBITION OF THE FORMATION OF AGEs

(75) Inventors: Nabil Abdul-Malak, Caluire (FR); Cecile Altobelli, Lyon (FR); Eric Perrier, Les Cotes D'Arey (FR)

Correspondence Address: BASF Beauty Care Solutions France;C/O Connolly Bove Lodge & Hutz LLP, 1007 North Orange Street, P.O. Box 2207, Wilmington, DE, 19899, US

(73) Assignee: BASF BEAUTY CARE SOLUTIONS FRANCE S.A.S., Lyon (FR)

(21) Appl. No.: 12/667,872

(22) PCT Filed: Jul. 9, 2008
(86) PCT No.: PCT/EP2008/058954

§ 371 Date: Mar. 10, 2010

Related U.S. Application Data
(60) Provisional application No. 61/078,910, filed on Jul. 8, 2008.

(30) Foreign Application Priority Data
Jul. 9, 2007 (FR) 0756343

(51) Int. Cl. A61K 36/00; A61K 36/13; A61K 36/73; A61K 31/352; A61K 36/45; A61K 36/20; A61K 31/122; A61K 31/7048; G01N 33/68; A61P 3/10

(52) U.S. Cl. 424/732; 424/725; 424/770; 424/765; 514/456; 424/771; 514/680; 514/27; 436/86

(57) ABSTRACT

The present invention relates to the use of an active substance that promotes the inhibition of the formation of AGEs, for preparing a composition to prevent and/or combat the reduction in elastic and plastic properties of tissues, and in particular of the skin, for inhibiting the formation of AGEs, or for preventing and/or combating glycation of proteins in the skin.

The invention also relates to a method of screening such active substances.

The invention relates to the use of at least one active principle for reducing the glycation of proteins capable of glycating in tissues, such as for example in the skin or in the tissue wall of a blood vessel or of an organ.

The present invention relates, in particular, to at least one substance that can be used topically or orally to act on the proteins capable of glycating in tissues.

### PRIOR ART

It is known in the prior art that non-enzymatic glycosylation or glycation is a purely chemical and spontaneous reaction that consists in covalently bonding a carbohydrate to a peptide chain.

Glycation is a fundamental mechanism of ageing that results from the attachment of free sugars to amino acids or to proteins.

Glycated proteins, also known as advanced glycation end products or AGEs, in particular reduce the flexibility, elasticity and functionality of the skin.

The glycation process occurs in three steps:
1—formation of a Schiff base resulting from the attachment of a reducing sugar (glucose or fructose) or of an aldehyde to the amino acid residues of the protein, mainly lysine and the N-terminal amine fraction.
2—Molecular rearrangement, known as Amadori rearrangement, which results from an isomerization of the Schiff base. The rate of formation of these Amadori products is proportional to the sugar concentration.
3—slow and irreversible accumulation, via rearrangements, hydrogen transfers and formation of very reactive intermediates, of glycation end products or Maillard products. This reaction leads to the formation of AGEs, better known by the expression Maillard products. The rate of formation of these compounds is independent of the sugar concentration of the medium that depends on the duration of hyperglycaemia and on the rate of protein turnover.

The first two stages (Schiff base and Amadori rearrangement) stabilize at a plateau and can be reversed depending on the level of glycaemia. On the other hand, the third step is irreversible and progresses regardless of the level of glycaemia.

Extracellular matrix proteins, the lifetime of which in the body is very long, are affected by glycation. Glycation modifies the properties of these proteins, making them more resistant to proteolysis and preventing their turnover. Furthermore, AGEs induce the formation of molecular bridges between the collagen fibres, making them more rigid and less soluble. Finally, the AGEs may have other actions by binding themselves to specific receptors present in macrophages, and endothelial and mesangial cells, by inducing the secretion of proinflammatory cytokines or growth factors. The importance of the glycation of proteins has been underlined by the effect of drugs which inhibit glycation, that is expressed by a slowing down of the ageing of certain functions in laboratory animals. In the course of diabetes mellitus, an excessive glycation of proteins also occurs, which is linked to the rise in glycaemia.

Aminoguanidine is a pharmaceutical substance derived from guanidine. It bonds to the glycation products by forming a reactive compound that is incapable of crosslinking. In the kidneys for example, it prevents the formation of AGEs in glomeruli and reduces the excretion of albumin in diabetics by 90%.

Patent Application US 2007/060533 describes the use of inhibitors of the formation of AGEs for the treatment of diabetes and various diseases linked to the formation of AGEs. This field is therefore not that of cosmetics where changes in the skin cannot reasonably be considered to be pathologies linked to the formation of AGEs. On the other hand, it should be noted that the anthocyanins are glycosylated.

Application US 2007/0003536 describes, in particular, the topical application of an extract of grape seed (vitis vinifera) in combination with phospholipids as free-radical scavengers, but does not describe an action on AGEs. The mechanisms are very different. The grape seed extract is described as being an anti-hydroxyl agent. Here too, there is no link with an activity on AGEs.

### OBJECTIVES OF THE INVENTION

The main objective of the invention is to solve the technical problem consisting of the provision of active substances that make it possible to limit the formation of AGEs resulting from the chemical glycation of proteins.

One particular objective of the present invention is to solve this technical problem within the context of the provision of cosmetic, dermatological or pharmaceutical compositions that prevent or combat the reduction in elastic and plastic properties of tissues, such as for example in the skin, or in the tissue wall of a blood vessel or of an organ, and in particular in the skin, especially during ageing of the tissues or during diabetes.

Another objective of the present invention is to provide a method of screening active principles that have the aforementioned properties.

One particular objective of the present invention is to solve the technical problem in a reliable and reproducible manner by providing non-toxic active substances, in particular for the cosmetic, dermatological, dermopharmaceutical or pharmaceutical industry, preferably that can be applied topically.

One particular objective of the invention is to provide active principles from plants, and especially to provide active principles that have low toxicity and are dermatologically acceptable.

Another objective of the present invention is to provide active principles for which the preparation is inexpensive and that can be produced on an industrial scale in a reliable and simple manner.

### DESCRIPTION OF THE INVENTION

Thus, the present invention describes the use of an active substance that promotes the inhibition of the formation of AGEs for preparing a composition to prevent and/or combat the reduction in elastic and plastic properties of tissues, such as for example in the skin, or in the tissue wall of a blood vessel or of an organ, and in particular in the skin.

The present invention relates to the use of an active substance for preparing a composition to promote the inhibition of the formation of AGEs.

The present invention relates to the use of an active substance for preparing a composition for preventing and/or combating the glycation of proteins in tissues, such as for example in the skin, or in the tissue wall of a blood vessel or of an organ.

Glycation is involved in numerous progressive diseases linked to ageing, such as vascular diseases (for instance, atherosclerosis), kidney disease, arthritis, complications of diabetes, cicatrization, etc. It is significant that the diabetic complications due to glycation can occur even at a younger age in diabetic individuals, whose average blood sugar concentration is higher than normal.

In diabetes, during which the involvement of free radicals is widely documented, oxidative stress is directly linked to hyperglycaemia. Sugars that are present in an too large amount in the blood then oxidize easily. This oxidation of the sugars leads, inter alia, to sugar/protein grafts or glycation. The increased level of glycated haemoglobin in the case of diabete is the typical example.

The glycation products, the level of which is proportional to the level of glycaemia, over a long period of time, are partly responsible for tissue ageing, the reduction in the elastic and plastic properties of which is one of the causes.

Thus, the present invention relates to the use of an active substance for preparing a composition to prevent and/or combat the glycation of proteins, especially in the skin, linked to the rise in glycaemia in the course of diabetes mellitus.

The present invention relates to the use of an active substance for preparing a composition for preventing and/or combating the formation of AGEs in glomeruli, and especially for reducing the excretion of albumin in diabetic subjects.

Advantageously, the composition is a cosmetic, dermopharmaceutical or pharmaceutical composition, preferably that can be applied topically or administered orally, for example as a food supplement.

The present invention also relates to a cosmetic composition, that can be applied topically or as a food (nutraceutical) supplement, for preventing and/or combating a reduction in elastic and plastic properties of tissues, in particular of the skin, such as in the case of combating and/or preventing ageing of a tissue, by inhibition of the formation of AGEs in the tissue, comprising, as an active substance, a substance that promotes the inhibition of the formation of AGEs, optionally in combination with a substance for promoting the protection of skin proteins such as aminoguanidine or guanidine, or an agent that inhibits glycation by mineralization, such as EDTA derivatives, phytic acid, azelaic acid, or an enzyme inhibitor such as pentosidine, or a substance capable of reducing the amount of sugar available to participate in the glycation reaction, such as carnosine, ascorbic acid or α-tocopherol.

The present invention also relates to a pharmaceutical composition, for example that can be applied topically or administered orally, especially intended to combat and/or prevent the reduction in elastic and plastic properties of a tissue, in particular of skin tissue, or of the tissue wall of a blood vessel or of an organ, by inhibiting the formation of AGEs in the tissue, characterized in that it comprises, as an active substance, a substance that promotes the inhibition of the formation of AGEs, optionally in combination with a substance for promoting the protection of tissue proteins, such as aminoguanidine or guanidine, or an agent that inhibits glycation by mineralization, such as EDTA derivatives, phytic acid, azelaic acid, or an enzyme inhibitor such as pentosidine, or a substance capable of reducing the amount of sugar available to participate in the glycation reaction, such as carnosine, ascorbic acid or α-tocopherol.

According to a first embodiment, the active substance that promotes the inhibition of the formation of AGEs is preferably chosen from an extract of a plant, a whole plant or part of a plant, chosen from an epimedium extract, an extract of curled dock, a sarsaparilla extract, an extract of indigenous vine (Davilla rugosa), guarana, preferably the seeds, a catechu extract, a milk thistle extract, a pine extract, an extract of Chinese rhubarb, a hawthorn extract, leucocyanidins, for example grape seed extracts, and in particular the leucocyanidins containing proanthocyanins of structure (I), an areca extract, a bilberry extract, an elder extract, a walnut extract, a willow extract, a lettuce extract, a lespedeza extract, and mixtures thereof.

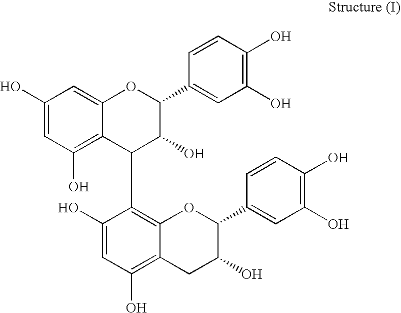

Depending on the plants, it is advantageous to use one part of the plant chosen from a root, rhizome, stem, bark, flower, fruit, seed, germ and leaf, preferably at 1-10% (w/w) in a solvent or a mixture of solvents, preferably a polar protic solvent, and advantageously in water, an alcohol, a glycol, a polyol, a water/alcohol, water/glycol or water/polyol mixture (such as water as a mixture with ethanol, glycerol, butylene glycol or other glycols, such as xylitol, etc.) from 100/0 to 0/100 (v/v). The extracts obtained are then preferably filtered or distilled in order to recover the soluble fraction which is then filtered. The active substance is advantageously the plant extract in a solvent, such as water, an alcohol, a polyol, a glycol or a mixture thereof, preferably diluted to a concentration between 0.01 and 10% (v/v).

The plant extracts according to the present invention are preferably chosen from the group consisting of:
epimedium (Epimedium brevicornum), preferably the leaves;
curled dock (Rumex crispus), preferably the root, the leaves and/or bark;
sarsaparilla (Smilax ornata); and
indigenous vine (Davilla rugosa), in particular its leaves.

The extract of indigenous vine (Davilla rugosa), in particular its leaves, is of broader interest for producing a cosmetic, pharmaceutical, in particular dermatological composition in combination with a suitable cosmetic and/or pharmaceutical, especially dermatological carrier. Such a composition is especially intended for topical application to prevent and/or combat skin ageing and its unattractive manifestations, in particular wrinkles, fine lines and grooves and makes it possible to improve the general condition of the skin. The indigenous vine is therefore useful for producing a cosmetic or dermatological care method consisting in applying the extract of indigenous vine to a part of the human body, preferably the skin, to improve the appearance and/or the condition thereof. The present invention thus relates to the use of this indigenous vine extract for cosmetic applications for an aesthetic purpose or for producing a pharmaceutical, especially dermatological, composition especially for protecting the skin against the harmful effects of environmental agents, especially pollutants and UV rays, and/or to repair it.

According to a second embodiment, the active substance that promotes the inhibition of the formation of AGEs is preferably chosen from the following characterized molecules: anthraquinone and derivatives thereof, preferably 1,4-anthraquinone and 1-amino-2-hydroxymethylanthraquinone, 4-hydroxychalcone, an OPC (or PCO) procyanidolic oligomer, such as OPC of the strawberry plant, leucocyanidins or proanthocyanins of structure (I), prunin, catechol, 4-aminophenol, sodium erythrosine, and also the plant extracts which contain them, and the mixtures thereof.

The characterized molecule according to the invention is preferably chosen from the group consisting of 1,4-anthraquinone, 1-amino-2-hydroxymethylanthraquinone, 4-hydroxychalcone, OPC of the strawberry plant, proanthocyanins of structure (I), prunin and mixtures thereof.

According to one particular embodiment, the active substance is the proanthocyanin of structure (I), preferably those obtained from grape seeds, in particular from an extract of the latter, said extract containing at least 90%, and preferably at least 95% by weight of proanthocyanins of structure (I). The invention therefore relates to a plant extract containing at least 90%, and preferably at least 95% by weight of the proanthocyanins of structure (I), and preferably obtained from grape seeds.

According to one particular embodiment, the OPC of the strawberry plant is obtained from the rhizome of the strawberry plant (Fragaria vesca), preferably in the form of an extract containing a mixture of oligomeric anthocyanidins, catechins, epicatechins and procyanidins, preferably in the form of dimers to hexamers.

Thus, the invention relates to the use, as an active ingredient that promotes the inhibition of the formation of AGEs, of a substance chosen among an extract of a plant, a whole plant or part of a plant, chosen from: guarana, catechu, milk thistle, pine, Chinese rhubarb, epimedium, hawthorn, leucocyanidins, areca, bilberry, elder, walnut, willow, curled dock, lettuce, sarsaparilla, indigenous vine and lespedeza; and/or a compound chosen among sodium erythrosine, 1,4-anthraquinone, catechol, 4-hydroxychalcone, 4-aminophenol, an OPC (PCO procyanidolic oligomer), such as OPC of the strawberry plant, or leucocyanidins or proanthocyanins of structure (I), prunin, and 1-amino-2-hydroxymethylanthraquinone, or a mixture of these substances, for preparing a cosmetic, nutraceutical or pharmaceutical composition.

Among all of these active substances, those which inhibit the formation of AGEs by at least 65%, and preferably by at least 80%, with reference to the inhibition produced by 1.5 mM aminoguanidine are preferred.

The preferred active ingredients in the compositions are chosen from the group consisting of
an extract of epimedium (Epimedium brevicornum), preferably the leaves, an extract of curled dock (Rumex crispus), preferably the root, the leaves and/or bark, an extract of sarsaparilla (Smilax ornata), preferably the roots, an extract of indigenous vine (Davilla rugosa), in particular the leaves, 1,4-anthraquinone, 4-hydroxychalcone, OPC of the strawberry plant, prunin or a combination of these.

The active substance may be concentrated by freeze drying, spray-drying, etc.

Advantageously, said substance is used at a concentration preferably between 0.001% and 10%, preferably between 0.01 and 5% and more particularly at 1% for the plant extracts, and preferably between 1×10⁻⁷ and 1%, preferably between 1×10⁻⁷ and 1×10⁻¹%, and more preferably between 1×10⁻⁵ and 1×10⁻¹% for the characterized molecules, by weight of the total composition, without this limiting the concentration to be used.

Thus, the present invention relates to a cosmetic care method comprising the topical application or ingestion in the form of a food (nutraceutical) supplement of a composition comprising, as a cosmetic active principle, at least one of the aforementioned active ingredients or active ingredients mentioned below.

The present invention also relates to a method of treating the human body comprising the administration of an aforementioned pharmaceutical composition or a pharmaceutical composition mentioned below, preferably topically, to prevent and/or combat the glycation of proteins of a tissue, especially when the blood sugar level rises and/or is high, such as for example during diabetes. The invention especially relates to a method of reducing the excretion of albumin in a diabetic subject.

The present invention also relates to a method of screening active principles that inhibit the formation of AGEs, comprising:
a) bringing at least one type of glycated protein into contact with a substance to be screened for its activity with respect to the inhibition of the formation of AGEs; and
b) the selection of at least one active principle that promotes the inhibition of the formation of AGEs.

Advantageously, step a) comprises an incubation of at least one type-of protein of the skin or of blood vessel walls, for example a collagen, or a bovine serum albumin with a reducing sugar (for example glucose, ribose or fructose) or an aldehyde under conditions that allow the formation of AGEs.

Advantageously, the skin protein is human collagen, which makes it possible to verify that the active substances of the invention are active on a protein predominantly in human skin.

Advantageously, step b) comprises the incubation of at least one type of protein of the skin or of blood vessel walls or of a bovine serum albumin with a reducing sugar (for example ribose, glucose or fructose) or of an aldehyde in the presence of at least one substance to be screened at a temperature between 40 and 60° C., preferably at around 50° C.

The selection of the active principle is carried out by comparison of the results obtained in the presence of the active agent tested relative to a positive control.

The compounds according to the present invention are prepared in the form of topical compositions, especially dermatologically acceptable cosmetic, dermopharmaceutical or pharmaceutical compositions. Therefore, for these compositions, the excipient contains, for example, at least one compound chosen from the group consisting of preservatives, emollients, emulsifiers, surfactants, moisturizers, thickeners, conditioners, matifying agents, stabilizers, antioxidants, texturizing agents, brighteners, film-forming agents, solubilizers, pigments, dyes, fragrances and sunscreens. These excipients are preferably chosen from the group consisting of amino acids and derivatives thereof, polyglycerols, esters, polymers and derivatives of cellulose, lanoline derivatives, phospholipids, lactoferrins, lactoperoxidases, sucrose-based stabilizers, vitamin E and derivatives thereof, natural and synthetic waxes, plant oils, triglycerides, unsaponifiable material, phytosterols, plant esters, silicones and derivatives thereof, protein hydrolysates, jojoba oil and derivatives thereof, fat-soluble/water-soluble esters, betaines, aminoxides, plant extracts, sucrose esters, titanium dioxides, glycines, and parabens, and more preferably from the group consisting of butylene glycol, steareth-2, steareth-21, glycol-15 stearyl ether, cetearyl alcohol, phenoxyethanol, methylparaben, ethylparaben, propylparaben, butylparaben, butylene glycol, natural tocopherols, glycerine, sodium dihydroxycetyl phosphate, isopropyl hydroxycetyl ether, glycol stearate, triisononanoin, octyl cocoate, polyacrylamide, isoparaffin, laureth-7, a carbomer, propylene glycol, glycerol, bisabolol, a dimethicone, sodium hydroxide, PEG-30 dipolyhydroxystearate, capric/caprylic triglycerides, cetearyl octanoate, dibutyl adipate, grape seed oil, jojoba oil, magnesium sulphate, EDTA, a cyclomethicone, xanthan gum, citric acid, sodium lauryl sulphate, mineral waxes and oils, isostearyl isostearate, propylene glycol dipelargonate, propylene glycol isostearate, PEG-8, beeswax, hydrogenated palm kernel oil glycerides, hydrogenated palm oil glycerides, lanoline oil, sesame oil, cetyl lactate, lanoline alcohol, castor oil, titanium dioxide, lactose, saccharose, low-density polyethylene and an isotonic saline solution.

Advantageously, the aforementioned compositions are formulated in a form chosen from the group consisting of an aqueous or oily solution, a cream or an aqueous gel or an oily gel, especially in a pot or in a tube, especially a shower gel or a shampoo; a milk; an emulsion, a microemulsion or a nanoemulsion, especially of the oil-in-water or water-in-oil or multiple or a silicone-based type; a lotion, especially, in a glass or plastic bottle or in a measuring bottle or in an aerosol; a bulb; a liquid soap; a dermatological cleansing bar; an ointment; a foam; an anhydrous product, preferably a liquid, pasty or solid product, for example in stick form, especially in the form of a lipstick.

The expression “topical application” used here means to apply or spray the composition according to the present invention over the surface of the skin.

The expression “dermatologically acceptable” used here means that the composition or the components of the latter are suitable for use in contact with human skin without undue toxicity, incompatibility, instability, allergic response or their equivalents.

The expression “that promotes the inhibition of the formation of AGEs” means that the substance makes it possible to reduce the formation of an advanced glycation end product (AGE) of a protein capable of glycating in the presence of a sugar. It is preferred that the substance promoting the inhibition of the formation of AGEs makes it possible to obtain, for example, a fluorescence of at least 65% of that obtained in the presence of 1.5 mM aminoguanidine and in the absence of the active substance that promotes the inhibition of the formation of AGEs, for example under the conditions of Example 1 below.

Many cosmetically active ingredients are known by a person skilled in the art for improving the health and/or the physical appearance of the skin. A person skilled in the art knows how to formulate the cosmetic or dermatological compositions to obtain the best effects. On the other hand, the compounds described in the present invention may have a synergistic effect when they are combined with one another. These combinations are also covered by the present invention. The CTFA Cosmetic Ingredient Handbook, Second Edition (1992) describes various cosmetic and pharmaceutical ingredients commonly used in the cosmetic and pharmaceutical industry, which are in particular suitable for topical use. Examples of these classes of ingredients comprise, without being limited to the following compounds: abrasives, absorbents, compounds having an aesthetic purpose such as fragrances, pigments, dyes, essential oils, astringents, etc. (for example: clove oil, menthol, camphor, eucalyptus oil, eugenol, menthyl lactate, witch hazel distillate), anti-acne agents, anti-flocculating agents, antifoaming agents, antimicrobial agents (for example: iodopropyl butylcarbamate), antioxidants, binders, biological additives, buffers, swelling agents, chelating agents, additives, biocides, denaturants, external analgesics, film-forming materials, polymers, opacifiers, pH adjusters, reducing agents, depigmenting or lightening agents (for example: hydroquinone, kojic acid, ascorbic acid, magnesium ascorbyl phosphate, ascorbyl glucosamine), conditioning agents (for example: humectants), skin-soothing agents and/or wound-healing agents (for example: panthenol and derivatives thereof (for example: ethyl panthenol), aloe vera, pantothenic acid and derivatives thereof, allantoin, bisabolol, and dipotassium glycyrrhizinate), thickeners, and vitamins, and derivatives or equivalents of the latter.

It is most particularly advantageous to combine the active substances that inhibit glycation with an active substance that makes it possible to limit the presence of AGEs, by reversing the Maillard reaction, by promoting the deglycation of AGEs, or promoting the reversion of the Maillard reaction with respect to AGEs, in particular those chosen from the group consisting of:
Aye wiwi (Maprounea guyanensis), and preferably the leaves;
Jamaica vervain (Stachytarpheta jamaicensis);
Cecropia (Cecropia obtuse), and preferably the leaves and/or the buds;
Pyrola (Chimaphila umbellata);
3,5-dimethoxy-4-hydroxycinnamic acid (sinapic acid);
trans-3,3′,4′,5,7-pentahydroxyflavane (catechin);
oxindole;
3,4-dihydroxyphenylacetic acid (DOPAC); and
1,3,5-trihydroxybenzene (phloroglucinol).

It is also particularly advantageous to combine the active substances according to the present invention with other active agents having complementary properties in order to further improve the efficacy against skin ageing, against reduction in flexibility and/or in plasticity and/or in elasticity and/or in functionality of the skin. Advantageously, these other active agents are chosen from:
active substances for stimulating cell proliferation and/or differentiation that have an anti-ageing effect, especially the following molecules: NGF, alpha-MSH, beta-endorphin or derivatives thereof, especially those described in Patent Application FR 2857874;
active substances protecting the fibroblast growth factor (FGF) especially FGF2, in particular those described in the Patent Application in the name of the Applicant published under the No. GB 244036, in particular an extract of Hibiscus Abelmoschus;
active substances stimulating the activity and/or proliferation of fibroblasts especially a fermented soy peptide, in particular that sold by the Applicant under the name Phytokine™, advantageously in combination with an extract of Hibiscus Abelmoschus;
active substances stimulating hyaluronase synthase, especially HAS2, in particular those described in Patent FR 2893252;
active substances stimulating the activity and/or the synthesis of lysyl oxidase, especially LOX, in particular those described in Patent Application FR 2855968, and preferably the dill extract; and
active substances that have antiinflammatory properties, such as that inhibiting PLA2, in particular an extract of Pueraria lobata roots such as described in Patent FR 2847267 (Inhipase®).

Other objectives, features and advantages of the invention will appear clearly to a person skilled in the art after reading the explanatory description which refers to examples which are given solely by way of illustration and should in no way limit the scope of the invention.

The examples are an integral part of the present invention and any feature that appears novel with respect to any prior art based on the description taken in its entirety, including the examples, is an integral part of the invention both functionally and generally.

Thus, each example has a general scope.

On the other hand, in the examples, all the percentages are given by weight, unless indicated otherwise, and the temperature is expressed in degrees Celsius unless indicated otherwise, and the pressure is atmospheric pressure, unless indicated otherwise.

### Examples

### Example 1

### Inhibition of the Formation of AGEs by Plant Extracts

1—Preparation of AGEs:

The preparation of a solution containing a protein and a reducing sugar was carried out extemporaneously as follows: a solution of bovine serum albumin (BSA) at a concentration between 1.5 μM and 1.5M, preferably between 15 μM and 500 μM, was incubated with a solution of a reducing sugar such as glucose, fructose, ribose, etc., preferably with glucose, at a concentration between 0.1M and 10M, preferably between 0.5M and 5M.

2—Preparation of the Potentially Active Principles of the “Plant Extract” Type:

The plant extracts were obtained by macerating the plants (preferably root, rhizomes, stems, bark, flowers, fruits, seeds, germs or leaves) at 1-10% (w/w) in a solvent or a mixture of solvents, advantageously chosen among water, an alcohol, a glycol, a polyol or a 100/0 to 0/100 (v/v) mixture of water and an alcohol, glycol or polyol (such as ethanol, glycerol, butylene glycol and other glycols, xylitol, etc.), and preferably in water. The extracts obtained were then filtered or distilled in order to recover the soluble fraction, which was then filtered, preferably to 0.45 μm, in order to obtain the extract to be tested.

3—Incubation of the AGEs with the Potentially Active Compound and Analysis of the Inhibition Obtained:

The protein/reducing sugar solution prepared in 1 was incubated with or without (negative control) the presence of an extract obtained in 2, at a temperature between 40 and 60° C., preferably at around 50° C. for 1 to 5 weeks, preferably for 3 weeks. The active substances obtained by extraction according to the protocol described in paragraph 2—extracts were tested at a concentration between 0.001 and 10%, preferably between 0.01 and 5% and more particularly at 1% by weight in the protein/reducing sugar preparation. The positive control used was aminoguanidine at a concentration between 15 μM and 150 mM, preferably at a concentration between 1.5 mM and 15 mM in water. The measure of the inhibition of the AGEs was carried out by measuring the fluorescence (excitation wavelength between 350 and 375 nm, preferably at 355 nm; emission wavelength between 420 and 450 nm, preferably at 430 nm). The inhibition was calculated by deducing the resulting value of the interaction of the plant extracts with the molecules of the reaction medium (quenching).

The plant extracts which inhibit the formation of AGEs under the conditions described were:

TABLE 1

| | | Part of | % inhi- |
|---|---|---|---|
| Common name | Latin name | the plant | bition |
| Guarana | Paullinia cupana | Seeds | 88.3 |
| Catechu | Acacia catechu | Wood | 87.4 |
| Milk thistle | Silybum marianum | Fruit | 86.4 |
| Pine | Pinus species | Root | 83.8 |
| Chinese rhubarb | Rheum officinale | Root | 82.3 |
| Pine | Pinus species | Bark | 80.1 |
| Epimedium | Epimedium brevicornum | Leaf | 79.9 |
| Hawthorn | Crataegus laevigata | Leaf | 79.0 |
| Leucocyanidins* | Vitis vinifera | Seeds | 78.4 |
| Areca | Areca catechu | Seeds | 77.4 |
| Bilberry | Vaccinium myrtillus | Fruit | 75.5 |
| Strawberry plant | Fragaria vesca | Rhizome | 74.8 |
| OPC | | | |
| Elderberry | Sambucus nigra | Fruit | 74.7 |
| Walnut | juglans regia | Leaf | 72.7 |
| Willow | salix alba | Bark | 71.8 |
| Curled dock | Rumex crispus | Bark | 69.9 |
| Lettuce | Lactuca sativa | Leaf | 69.4 |
| Sarsaparilla | Smilax ornata | Root | 66.3 |
| Lespedeza | Lespedeza capitata | Leaf | 65.1 |
| Indigenous vine | Davilla rugosa | Leaf | 52.0 |

% inhibition = fluorescence obtained with the active principle versus the fluorescence obtained with the positive control (1.5 mM aminoguanidine) in %.
*The leucocyanidins of the present invention were an aqueous extract (preferably by maceration in water) starting from grape seeds and contained 95% of proanthocyanins corresponding to the following structure (I):

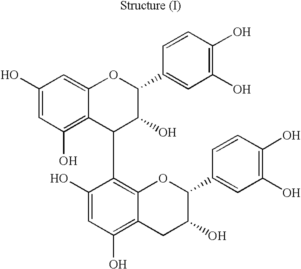

By way of example, mention may be made of the leucocyanidins recorded under No. 84929-27-1.

### Example 2

### Inhibition of the Formation of AGEs by the Characterized Molecules

The preparation of a protein/reducing sugar solution was carried out according to the method described in Example 1 (paragraph 1). The molecules were tested at a concentration between 1×10⁻⁷ and 1×10⁻¹%, and preferably between 1×10⁻⁵ and 1×10⁻¹%, and in particular at 1×10⁻¹%, for example in water or in DMSO.

The molecules which inhibit the formation of AGEs under the conditions described were:

TABLE 2

| Common name | INCI | CAS | % inhibition |
|---|---|---|---|
| Sodium erythrosine | Aka or CI | 1342-25-2 or | 95.6 |
| | 45430 or Acid | 16423-68-0 | |
| | Red 51 | | |
| 1,4-Anthraquinone | | 635-12-1 | 89.6 |
| Catechol | Pyrocatechol | 120-80-9 | 88.3 |
| 4-Hydroxychalcone | | 20426-12-4 | 81.4 |
| 4-Aminophenol | p-Aminophenol | 123-30-8 | 78.5 |
| Prunin | No CTFA filing | | 67.9 |
| 1-Amino-2-hydroxy- | No CTFA filing | | 66.0 |
| methylanthraquinone | | | |

% inhibition = fluorescence obtained with the active principle versus the fluorescence obtained with the positive control (1.5 mM aminoguanidine) in %.

### Example 3

### Activity of the Substances According to the Present Invention on the Anti-Glycation of Human Collagen

The preparation of a protein/reducing sugar solution was carried out according to the method described in Example 1 (paragraph 1) with the use of human collagen replacing BSA.

1—Preparation of Human Collagen.

The extraction of human collagen was carried out, from a human biopsy resulting from plastic surgery. The collagen obtained in the form of a solution was incubated with a solution of a reducing sugar such as glucose, fructose or ribose, and preferably with ribose. The incubation concentrations were the same as those described in the preceding examples.

2—Preparation and Test of the Potentially Active Principles

The potentially active principles of the “plant extract” type and of the “characterized molecule” type were used under the same conditions as those cited in Examples 1 and 2.

3—Results

The molecules that inhibit the formation of AGEs under the conditions described, in a manner at least equivalent to that of the 1.5 mM aminoguanidine used, were the same as those cited in Examples 1 and 2.

TABLE 3
Comparative results between BSA and human
collagen (plant extract at 5%)

| | | Inhibition with |
|---|---|---|
| | Inhibition with | human |
| | BSA/glucose | collagen/ribose |
| | versus 1.5 mM | versus 1.5 mM |
| Name | aminoguanidine | aminoguanidine |
| Leucocyanidins* | 70% | 77% |
| Sarsaparilla | 52% | 52% |
| Milk thistle | 71% | 90% |

*See above.

### Example 4

### Comparison Between Proanthocyanidins According to the Present Invention and Certain Anthocyanidins from the Prior Art

In this example, the inventors desire to study the properties of inhibition of the formation of AGEs for various molecules from the family of anthocyanins.

TABLE 4

| Common name | INCI | CAS | % inhibition |
|---|---|---|---|
| Leucocyanidins* | Grape (Vitis | // | 78.4 |
| | vinifera) Seed | | |
| | Extract | | |
| Perlargonin | — | 17334-58-6 | 0 |
| chloride | | | |
| Idaein chloride | — | 27661-36-5 | 0 |
| Malvin Chloride | — | 16727-30-3 | 0 |
| Kuromanin Chloride | — | 7084-24-4 | 0 |

% inhibition: fluorescence obtained with the active principle versus the fluorescence obtained with the positive control (1.5 mM aminoguanidine) in %.
*See above.

It has surprisingly been discovered that the grape seed extract containing 95% of proanthocyanidins of structure (I) according to the present invention had a very high activity compared to the glycosylated molecules of the prior art. In the prior art, various anthocyanins had been reported as being active for inhibiting the formation of AGEs. However this observation does not generally take into account the “quenching” or noise of the signal generated by the absorption properties of certain wavelengths of the anthocyanins themselves, and is not connected to an inhibitory activity for the formation of AGEs.

In the present invention, the “quenching” is duly taken into account. Thus the results observed can be directly attributed to the inhibition of the formation of AGEs.

### Example 5

### Comparison with Respect to the Teaching of Patent Application US 2007/0060533 by Yohikawa et al., “Novel Inhibitor of the Formation of Advanced Glycation End Product and aldose Reductase Inhibitor”

The anthocyanins from document US 2007/0060533, which may be used as inhibitors and can be obtained by extraction making it possible to obtain large amounts of anthocyanins, are especially derived from berries.

The inventors have tested the following berries in comparison with the leucocyanidins of the invention:

TABLE 5

| | Inhibition versus 1.5 mM |
|---|---|
| Name of the berries | aminoguanidine |
| Leucocyanidins* | 78% |
| Bilberry | 39% |
| Cranberry | 33% |
| Raspberry | 0% |
| Plum | 0% |
| Elderberry | 0% |
| Hibiscus | 0% |
| Blackberry | 0% |
| Redcurrant | 0% |
| Red cabbage | 0% |
| Strawberry | 0% |

*See above.

It is observed that the leucocyanidins of the invention have an activity much higher than that of the anthocyanins which are contained in the active berries according to the prior art.

### Examples 6 to 11

### Anti-Ageing Composition that can be Applied Topically

In the following examples, “products of the invention” represent the active substances according to the invention and in particular those obtained according to Example 1 or 2.

### Example 6

### Use of the Products of the Invention in Cosmetic or Pharmaceutical Formulations of the Oil-In-Water Emulsion Type

Formulation 6a:

| A | Water | qs for 100 |
|---|---|---|
| | Butylene Glycol | 2 |
| | Glycerine | 3 |
| | Sodium Dihydroxycetyl | 2 |
| | Phosphate, | |
| | Isopropyl Hydroxycetyl Ether | |
| B | Glycol Stearate SE | 14 |
| | Triisononaoin | 5 |
| | Octyl Cocoate | 6 |
| C | Butylene Glycol, | 2 |
| | Methylparaben, | |
| | Ethylparaben, Propylparaben | |
| | pH adjusted to 5.5 | |
| D | Products of the invention | 0.001-10% |

Formulation 6b:

| A | Water | qs for 100 |
|---|---|---|
| | Butylene Glycol | 2 |
| | Glycerine | 3 |
| | Polyacrylamide, Isoparaffin, | 2.8 |
| | Laureth-7 | |
| B | Butylene Glycol, | 2 |
| | Methylparaben, | |
| | Ethylparaben, Propylparaben; | |
| | Phenoxyethanol, | 2 |
| | Methylparaben, | |
| | Propylparaben, Butylparaben, | |
| | Ethylparaben | |
| | Butylene Glycol | 0.5 |
| D | Products of the invention | 0.001-10% |

Formulation 6c:

| | | |
|---|---|---|
| A | Carbomer | 0.50 |
| | Propylene Glycol | 3 |
| | Glycerol | 5 |
| | Water | qs for 100 |
| B | Octyl Cocoate | 5 |
| | Bisabolol | 0.30 |
| | Dimethicone | 0.30 |
| C | Sodium Hydroxide | 1.60 |
| D | Phenoxyethanol, | 0.50 |
| | Methylparaben, | |
| | Propylparaben, Butylparaben, | |
| | Ethylparaben | |
| E | Fragrance | 0.30 |
| F | Products of the invention | 0.001-10% |

### Example 7

### Use of the Products of the Invention in a Formulation of the Water-In-Oil Type

| A | PEG-30 | |
|---|---|---|
| | dipolyhydroxystearate | 3 |
| | Capric Triglycerides | 3 |
| | Cetearyl Octanoate | 4 |
| | Dibutyl Adipate | 3 |
| | Grape Seed Oil | 1.5 |
| | Jojoba Oil | 1.5 |
| | Phenoxyethanol, | 0.5 |
| | Methylparaben, | |
| | Propylparaben, Butylparaben, | |
| | Ethylparaben | |
| B | Glycerine | 3 |
| | Butylene Glycol | 3 |
| | Magnesium Sulphate | 0.5 |
| | EDTA | 0.05 |
| | Water | qs for 100 |
| C | Cyclomethicone | 1 |
| | Dimethicone | 1 |
| D | Fragrance | 0.3 |
| E | Products of the invention | 0.001-10% |

### Example 8

### Use of the Products of the Invention in an Aqueous Gel (Eye Contour, etc.) Formulation

| A | Water | qs for 100 |
|---|---|---|
| | Carbomer | 0.5 |
| | Butylene Glycol | 15 |
| | Phenoxyethanol, Methylparaben, | 0.5 |
| | Propylparaben, Butylparaben, | |
| | Ethylparaben | |
| B | Products of the invention | 0.001-10% |

### Example 9

### Use of the Products of the Invention in a Shampoo or Shower Gel Type Formulation

| | | |
|---|---|---|
| A | Xanthan Gum | 0.8 |
| | Water | qs for 100 |
| B | Butylene Glycol, | 0.5 |
| | Methylparaben, | |
| | Ethylparaben, Propylparaben | |
| | Phenoxyethanol, | 0.5 |
| | Methylparaben, | |
| | Propylparaben, Butylparaben, | |
| | Ethylparaben | |
| C | Citric acid | 0.8 |
| D | Sodium Laureth Sulphate | 40.0 |
| E | Product of the invention | 0.001-10% |

### Example 10

### Use of the Products of the Invention in a Lipstick and Other Anhydrous Products Type Formulation

| | | |
|---|---|---|
| A | Mineral Wax | 17.0 |
| | Isostearyl Isostearate | 31.5 |
| | Propylene Glycol Dipelargonate | 2.6 |
| | Propylene Glycol Isostearate | 1.7 |
| | PEG 8 Beeswax | 3.0 |
| | Hydrogenated Palm Kernel Oil | 3.4 |
| | Glycerides, | |
| | Hydrogenated Palm Glycerides | |
| | Lanolin Oil | 3.4 |
| | Sesame Oil | 1.7 |
| | Cetyl Lactate | 1.7 |
| | Mineral Oil, Lanolin Alcohol | 3.0 |
| B | Castor Oil | qs for 100 |
| | Titanium Dioxide | 3.9 |
| | CI 15850:1 | 0.616 |
| | CI 45410:1 | 0.256 |
| | CI 19140:1 | 0.048 |
| | CI 77491 | 2.048 |
| C | Products of the invention | 0.001-5% |

### Example 11

### Use of the Products of the Invention in a Tablet, Ointment or Injectable Formulation

Formulation 11a: Preparation of Tablets

| A | Excipients | in g per tablet |
|---|---|---|
| | Lactose | 0.359 |
| | Sucrose | 0.240 |
| B | Active principle* | 0.001-0.1 |

*The active principle is obtained, for example, according to the extraction process described in the examples, followed by a drying step.

Formulation 11b: Preparation of an Ointment

| A | Excipients | |
|---|---|---|
| | Low-density polyethylene | 5.5 |
| | Liquid paraffin | qs for 100 |
| B | Active principle* | 0.001-0.1 |

*The active principle is obtained, for example, according to the extraction process described in the examples, followed by a drying step.

Formulation 11c: Preparation of an Injectable Formula

| A | Excipient | | |
|---|---|---|---|
| | Isotonic saline solution | 5 | ml |
| B | Active principle* | 0.001-0.1 | g |

*The active principle is obtained, for example, according to the extraction process described in the examples, followed by a drying step.

Phase A and phase B are packaged in separate ampoules and mixed before use.

1-14. (canceled)

15. A method of promoting the the inhibition of the formation of AGEs in tissues comprising the application or ingestion of a cosmetic, nutraceutical, or pharmaceutical composition comprising an active substance capable or inhibiting the formation of AGEs said substance selected from the group consisting of epimedium extract, curled dock extract, sarsaparilla extract, indigenous vine extract, guarana seed extract, catechu extract, milk thistle extract, pine extract, Chinese rhubarb extract, hawthorn extract, leucocyanidins, areca extract, bilberry extract, elder extract, walnut extract, willow extract, lettuce extract, sarsaparilla extract, lespedeza extract, anthraquinone and derivatives thereof, 4-hydroxychalcone, an OPC (PCO procyanidolic oligomer), leucocyanidins or proanthocyanins of structure (I), 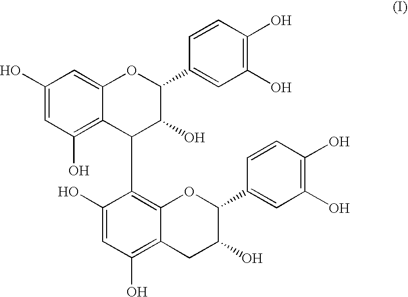
prunin, catechol, 4-aminophenol, sodium erythrosine, and combinations thereof.

16. The method of claim 15 wherein the active substance is selected from the group consisting of a leaf extract of epimedium (Epimedium brevicornum), a root, leaf or bark extract of curled dock (Rumex crispus), a root extract of sarsaparilla (Smilax ornata), a leaf extract of indigenous vine (Davilla rugosa), 1,4-anthraquinone, 4-hydroxychalcone, OPC of the strawberry plant, prunin and combinations thereof.

17. The method of claim 15 wherein the composition is a cosmetic, nutraceutical or pharmaceutical composition for preventing and/or combating the reduction in the elastic properties of a tissue by inhibiting the formation of AGEs or for preventing and/or combating glycation of proteins in the tissue.

18. The method of claim 15 wherein said substance is used at a concentration between 0.001 and 10% for the plant extracts and between 1×10⁻⁷ and 1% for the characterized molecules, by weight of the total composition.

19. A cosmetic composition for preventing or combating the reduction in the elastic properties of the skin by inhibition of the cutaneous formation of AGEs comprising an active substance selected from the group consisting of epimedium extract, curled dock extract, sarsaparilla extract, indigenous vine extract, guarana seed extract, catechu extract, milk thistle extract, pine extract, Chinese rhubarb extract, hawthorn extract, leucocyanidins, areca, bilberry extract, elder extract, walnut extract, willow extract, lettuce extract, lespedeza extract, an anthraquinone and derivatives thereof, 4-hydroxychalcone, an OPC (PCO procyanidolic oligomer), leucocyanidins or proanthocyanins of structure (I) 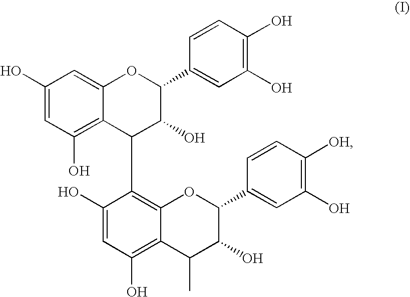
prunin, catechol, 4-aminophenol, sodium erythrosine, and combinations thereof.

20. The composition according to claim 19, in which the active substance is chosen among the group consisting of a leaf extract of epimedium (Epimedium brevicornum), a root, leaf or bark extract of curled dock (Rumex crispus), a root extract of sarsaparilla (Smilax ornata), a leaf extract of indigenous vine (Davilla rugosa), 1,4-anthraquinone, 4-hydroxychalcone, OPC of the strawberry plant, prunin and combinations thereof.

21. The composition according to claim 19 wherein said substance is used at a concentration between 0.001 and 10% for the plant extracts and between 1×10⁻⁷ and 1% for the characterized molecules, by weight of the total composition.

22. A pharmaceutical composition for combating and/or preventing the reduction in the elastic properties of a tissue by inhibiting the formation of AGEs in the tissue comprising an active substance that promotes the inhibition of the formation of AGEs selected from the group consisting of epimedium extract, curled dock extract, sarsaparilla extract, indigenous vine extract, guarana seed extract, catechu extract, milk thistle extract, pine extract, Chinese rhubarb extract, hawthorn extract, leucocyanidins, areca extract, bilberry extract, elder extract, walnut extract, willow extract, lettuce extract, sarsaparilla extract, lespedeza extract, anthraquinone and derivatives thereof, an OPC (PCO procyanidolic oligomer), leucocyanidins or proanthocyanins of structure (I) 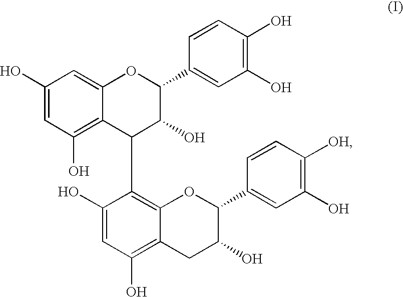
prunin, catechol, 4-aminophenol, sodium erythrosine, and combinations thereof.)

23. The composition according to claim 22 wherein the active substance is chosen among the group consisting of a leaf extract of epimedium (Epimedium brevicornum), a root, leaf, or bark extract of curled dock (Rumex crispus), a root extract of sarsaparilla (Smilax ornata), a leaf extract of indigenous vine (Davilla rugosa), 1,4-anthraquinone, 4-hydroxychalcone, OPC of the strawberry plant, prunin and combinations thereof.

24. The composition according to claim 8 wherein the substance is used at a concentration between 0.001 and 10% for the plant extracts and between 1×10⁻⁷ and 1% for the characterized molecules, by weight of the total composition.

25. A method of screening active substances that inhibit the formation of AGEs, comprising:
a) bringing at least one type of glycated protein into contact with a substance to be screened for its activity with respect to the inhibition of the formation of AGEs; and
b) the selection of at least one active substance that promotes the inhibition of the formation of AGEs.

26. The screening method of claim 25 wherein step a) further comprises an incubation of at least one type of protein of the skin or of blood vessel walls with a reducing sugar or BSA (bovine serum albumin) with a reducing sugar under conditions that allow the formation of AGEs.

27. The screening method of claim 25 wherein step b) further comprises the incubation of at least one type of protein of the skin or of blood vessel walls, for example a human collagen, with a reducing sugar or BSA (bovine serum albumin), with a reducing sugar in the presence of at least one substance to be screened at a temperature between 40° C. and 60° C.
